(19) United States
(12) Patent Application Publication
(10) Pub. No.: US 2010/0010261 A1
(43) Pub. Date: Jan. 14, 2010

(54) PRODUCTION OF AMINES

(75) Inventors: Graham Ronald Eastham, Redcar (GB); David Cole-Hamilton, Fife (GB); Angel Alberto Nunez Magro, Fife (GB)

Correspondence Address: VENABLE LLP, P.O. BOX 34385, WASHINGTON, DC, 20043-9998, US

(73) Assignee: LUCITE INTERNATIONAL UK LIMITED, Hampshire (GB)

(21) Appl. No.: 12/442,315

(22) PCT Filed: Sep. 21, 2007
(86) PCT No.: PCT/GB07/50571

§ 371 Date: Jul. 1, 2009

(30) Foreign Application Priority Data
Sep. 22, 2006 (GB) 0618649.8
Mar. 24, 2007 (GB) 0705713.6

(51) Int. Cl. C07C 231/12; C07C 231/02

(52) U.S. Cl. 564/192

(57) ABSTRACT

A process for the hydrogenation of carboxylic acids and/or derivatives, particularly amides, is described. The process includes reacting an acid or derivative such as an amide with a source of hydrogen in the presence of a catalyst system. The catalyst system obtainable by combining: (a) a source of ruthenium, and (b) a phosphine compound of general Formula I: (Formula I). The hydrogenation reaction is carried out in the presence of a low concentration of water or at low pressure or in the presence of a source of ammonia or the hydrogenation reaction is carried out in the absence of water or a combination of these factors is utilised. The invention also relates to the use of ammonia in the production of primary amines by hydrogenation of carboxylic acids and/or derivatives thereof or a process for the production of primary amines generally.

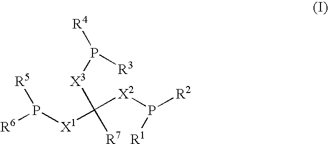

The present invention relates to the hydrogenation of carboxylic acids, and/or derivatives such as esters and amides, to amines, more specifically, the homogeneously catalysed hydrogenation of such acids, esters and/or amides to amines.

Prior art documents disclose the use of heterogeneous catalysts to catalyse hydrogenation reactions. For example, JP 2001-226327 discloses the hydrogenation of aliphatic nitriles to amines using a nickel catalyst. WO 98/03262 discloses the preparation of amines from fatty amides using an optionally metal-promoted copper chromite catalyst.

WO 03/093208 discloses a homogeneous process for the hydrogenation of carboxylic acids and derivatives thereof in the presence of a catalyst comprising ruthenium and an organic phosphine to give a secondary amine in low yield.

It has now surprisingly been found that catalysing the hydrogenation of carboxylic acids and/or derivatives such as esters and amides with a specific catalyst system leads to a highly selective conversion resulting in a high yield of the desired amine product.

Furthermore, it has also been surprisingly found that a primary amine may be selectively produced in high yield from the above hydrogenation system in the presence of ammonia.

According to a first aspect of the present invention, there is provided a process for the hydrogenation of carboxylic acids and/or derivatives thereof comprising the steps of:—
reacting said carboxylic acids and/or derivatives thereof with a source of hydrogen in the presence of a catalyst system, the said catalyst system obtainable by combining:
   (a) a source of ruthenium, and
   (b) a phosphine compound of general formula I:

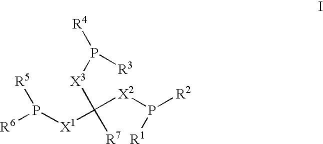

wherein X¹ to X³ and R¹ to R⁶ each independently represent lower alkyl or aryl, and R⁷ represents hydrogen, lower alkyl or aryl,
   wherein the hydrogenation reaction is carried out in the presence of a low concentration of water or in the absence of water.

According to a second aspect of the present invention, there is provided a process for the hydrogenation of carboxylic acids and/or derivatives thereof comprising the steps of:—
reacting said carboxylic acids and/or derivatives thereof with a source of hydrogen in the presence of a catalyst system, the said catalyst system obtainable by combining:
(a) a source of ruthenium, and
(b) a phosphine compound of general formula I:

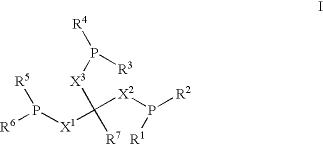

wherein X¹ to X³ and R¹ to R⁶ each independently represent lower alkyl or aryl, and R⁷ represents hydrogen, lower alkyl or aryl,
   wherein the reaction is carried out at a low pressure.

Preferably, the catalyst system is homogeneous.

By the term “homogeneous” we mean a catalyst system wherein the catalyst is in the same phase as the reactants. For example, wherein the catalyst is not supported but is simply admixed or formed in-situ with the reactants of the hydrogenation reaction, preferably in a suitable solvent as described herein.

Preferably, the step of reacting said carboxylic acids and/or derivatives thereof with a source of hydrogen in the presence of a homogenous catalyst system is carried out in the presence of at least one solvent. Any suitable solvent may be used. Such suitable solvents will be able to dissolve the catalyst system and hold the catalyst system in phase with the amide. Examples of suitable solvents include ethereal solvents including ethers such as diethyl ether, and dioxane; organic solvents such as toluene, benzene and xylene; heterocyclic organic solvents such as tetrahydrofuran.

An especially preferred solvent for use in the present invention is tetrahydrofuran (THF).

It has surprisingly been found that a very high conversion of carboxylic acids and/or derivatives thereof to the desired hydrogenation products is obtained when using the process of the present invention in the presence of a low concentration of water.

It is preferable, therefore, for the hydrogenation reaction to occur under low concentrations of water. A lower concentration of water in the reaction mixture leads to an increase in the conversion of carboxylic acid and/or derivative thereof to the desired products in the hydrogenation reaction.

Preferably, the ratio of moles of water:moles of ruthenium present at the start of a batch reaction or during a continuous reaction is up to about 2500:1, preferably up to about 2000:1, more preferably up to about 1500:1.

Preferably, the ratio of moles of water:moles of ruthenium present at the start of a batch reaction or during a continuous reaction is at least about 50:1, preferably at least about 100:1, more preferably at least about 200:1.

Preferably, the ratio of the volume of water:volume of solvent present in the reaction is up to about 4:10, preferably, up to about 2:10, most preferably, up to about 1:10. The reaction may proceed in an absence of water. In this case, a full conversion of an amide to an amine may be obtained, with only traces of alcohol produced. However, the catalyst may not always be stable under these conditions. Therefore, it may be beneficial to provide a minimal amount of water to increase stability of the catalyst, while allowing for a good conversion of the carboxylic acid and/or derivative thereof.

By present in the reaction, is meant present at any time during the reaction, preferably, present in the reaction at the start of a batch process or during a continuous process.

The desired amount of water may be added to the reaction mixture prior to the hydrogenation reaction in a batch process or during a continuous process.

In one embodiment, the water present in the reaction mixture may be added in the form of aqueous ammonia.

It has further been surprisingly found that a very high conversion of carboxylic acid and/or derivative thereof to the desired product is obtained when the process of the present invention is carried out under a low pressure. Therefore, it is advantageous for the hydrogenation reaction to occur under low pressures.

The reaction may be carried out under a pressure of up to about 6.5×10⁶ Pa, preferably up to about 5.0×10⁶ Pa, and most preferably up to about 4.0×10⁶ Pa.

Preferably, the source of hydrogen is hydrogen gas. The hydrogen gas may be used either in pure form or diluted with one or more inert gases, such as nitrogen, carbon dioxide and/or a noble gas such as argon.

Preferably, the pressure under which the reaction is carried out is provided by the pressure of the source of hydrogen and any other gas which is present in the hydrogen gas. The total gaseous pressure of the source of hydrogen and any other gas present may, therefore, be up to about 6.5×10⁶ Pa, preferably up to about 5.0×10⁶ Pa, and most preferably up to about 4.0×10⁶ Pa.

The source of ruthenium used in the catalyst system of the present invention may be in the form of a ruthenium salt. Salts of ruthenium that may be useful in the present invention include those that may be converted into active species under the hydrogenation reaction conditions. Such salts include nitrates, sulphates, carboxylates, beta diketones, carbonyls and halides.

Specific examples of suitable sources of ruthenium include, but are not limited to, any of the following: ruthenium nitrate, ruthenium dioxide, ruthenium tetraoxide, ruthenium dihydroxide, ruthenium acetylacetonate, ruthenium acetate, ruthenium maleate, ruthenium succinate, tris-(acetylacetone)ruthenium, pentacarbonylruthenium, dipotassium tetracarbonylruthenium, cyclo-pentadienyldicarbonyltrithenium, tetrahydridedecacarbonyltetraruthenium, tetraphenylphosphonium, ruthenium dioxide, ruthenium tetraoxide, ruthenium dihydroxide, bis(tri-n-butylphosphine)tricarbonylruthenium, dodecacarbonyl-triruthenium, tetrahydridedecacarbonyltetraruthenium, tetraphenylphosphonium, undecacarbonylhydridetriruthenate.

An especially preferred source of ruthenium for use in the present invention is tris-(acetylacetone)ruthenium (Ru(acac)₃).

Any suitable phosphine of general formula I may be used. Preferably, X¹ to X³ in formula I each independently represent a divalent bridging group. Preferably, X¹ to X³ in formula I each independently represent lower alkylene or arylene. More preferably, X¹ to X³ each independently represent C₁ to C₆ alkylene, which may be optionally substituted as defined herein, or phenylene (wherein the phenylene group may be optionally substituted as defined herein). Even more preferably, X¹ to X³ each independently represent C₁ to C₆ alkylene, which may be optionally substituted as defined herein. Most preferably, X¹ to X³ each independently represent non-substituted C₁ to C₆ alkylene such as methylene, ethylene, n-propylene, iso-propylene, n-butylene, iso-butylene, pentylene, hexylene or cyclohexylene. An especially preferred non-substituted C₁ to C₆ alkylene is methylene.

In an especially preferred embodiment of the present invention each X¹ to X³ group represents the same lower alkylene or arylene group as defined herein. Preferably, when alkylene groups, each X¹ to X³ represents the same C₁ to C₆ alkylene group, particularly non-substituted C₁-C₆ alkylene, such as methylene, ethylene, n-propylene, iso-propylene, n-butylene, iso-butylene, pentylene, hexylene or cyclohexylene. More preferably, each X¹ to X³ represents methylene.

Preferably, R¹ to R⁶ in formula I each independently represent lower alkyl or aryl groups. More preferably, R¹ to R⁶ each independently represent C₁ to C₆ alkyl, which may be optionally substituted as defined herein, or phenyl (wherein the phenyl group may be optionally substituted as defined herein). Most preferably, R¹ to R⁶ each independently represent a non-substituted C₁ to C₆ alkyl such as methyl, ethyl, n-propyl, iso-propyl, n-butyl, iso-butyl, tert-butyl, pentyl, hexyl or cyclohexyl or phenyl. An especially preferred group is phenyl.

In an especially preferred embodiment of the present invention each R¹ to R⁶ group represents the same lower alkyl or aryl group as defined herein. More preferably, each R¹ to R⁶ represents a non-substituted C₁ to C₆ alkyl such as methyl, ethyl, n-propyl, iso-propyl, n-butyl, iso-butyl, tert-butyl, pentyl, hexyl or cyclohexyl or phenyl. Most preferably, each R¹ to R⁶ represents phenyl.

Preferably, R⁷ in formula I represents hydrogen, lower alkyl or aryl. More preferably, R⁷ represents H or C₁ to C₆ alkyl, which may be optionally substituted as defined herein. Most preferably, R⁷ represents H or non-substituted C₁ to C₆ alkyl such as methyl, ethyl, n-propyl, iso-propyl, n-butyl, iso-butyl, tert-butyl, pentyl or hexyl. Especially preferred groups are H or methyl.

Specific examples of phosphine compounds of general formula I include, but are not limited to, tris-1,1,1-(diphenylphosphinomethyl)methane, tris-1,1,1-(diphenylphosphinomethyl)-ethane, tris-1,1,1-(diphenylphosphinomethyl)propane, tris-1,1,1-(diphenylphosphinomethyl)butane, tris-1,1,1-(diphenylphosphinomethyl)-2-ethane-butane, tris-1,1,1-(diphenylphosphinomethyl)2,2-dimethylpropane, tris-1,1,1-(dicyclohexylphosphinomethyl)ethane, tris-1,1,1-(dimethylphosphinomethyl)ethane and tris-1,1,1-(diethylphosphinomethyl)ethane.

An especially preferred phosphine compound is 1,1,1-tris(diphenylphosphinomethyl)ethane (also known as triphos).

The term “lower alkyl” when used herein, means C₁ to C₁₀ alkyl and includes methyl, ethyl, ethenyl, propyl, propenyl butyl, butenyl, pentyl, pentenyl, hexyl, hexenyl and heptyl groups. Unless otherwise specified, alkyl including lower alkyl groups may, when there is a sufficient number of carbon atoms, be linear or branched, be saturated or unsaturated, be cyclic, acyclic or part cyclic/acyclic, be unsubstituted, substituted or terminated as defined herein and/or be interrupted by one or more (preferably less than 4) oxygen, sulphur, silicon atoms, or by silano or dialkylsilicon groups, or mixtures thereof.

The term “substituted” herein means, unless otherwise defined, substituted or terminated by one or more substituents selected from halo, cyano, nitro, OR¹⁹, OC(O)R²⁰, C(O)R²¹, C(O)OR²², NR²³R²⁴, C(O)NR²⁵R²⁶, SR²⁹, C(O)SR³⁰, C(S)NR²⁷R²⁸, unsubstituted or substituted aryl, lower alkyl (which group may itself be unsubstituted or substituted or terminated as defined herein), or unsubstituted or substituted Het, wherein R¹⁹ to R³⁰ each independently represent hydrogen, unsubstituted or substituted aryl or unsubstituted or substituted lower alkyl. Preferably, when the substituent is itself substituted, the further substituent terminates the substituent.

The term “alkylene” as used herein, relates to a bivalent radical alkyl group otherwise defined as lower alkyl above. For example, an alkyl group such as methyl which would be represented as —CH₃, becomes methylene, —CH₂—, when represented as an alkylene. Other alkylene groups should be understood accordingly.

The term “aryl” when used herein, includes five-to-ten-membered, preferably six to ten membered, carbocyclic aromatic or pseudo aromatic groups, such as phenyl, ferrocenyl and naphthyl, which groups may be unsubstituted or substituted with one or more substituents selected from unsubstituted or substituted aryl, lower alkyl (which group may itself be unsubstituted or substituted or terminated as defined herein), Het (which group may itself be unsubstituted or substituted or terminated as defined herein), halo, cyano, nitro, OR¹⁹, OC(O)R²⁰, C(O)R²¹, C(O)OR²², NR²³R²⁴, C(O)NR²⁵R²⁶, SR²⁹, C(O)SR³⁰ or C(S)NR²⁷R²⁸ wherein R¹⁹ to R³⁰ each independently represent hydrogen, unsubstituted or substituted aryl or lower alkyl (which alkyl group may itself be unsubstituted or substituted or terminated as defined herein).

The term “arylene” as used herein, relates to a bivalent radical aryl group as otherwise defined above. For example, an aryl group such as phenyl which would be represented as —PH, becomes phenylene, —PH—, when represented as an arylene. Other arylene groups should be understood accordingly.

Halo groups with which the above-mentioned groups may be substituted or terminated include fluoro, chloro, bromo and iodo.

The term “Het”, with which the above-mentioned groups may be substituted or terminated, includes four- to twelve-membered, preferably four- to ten-membered ring systems, which rings contain one or more heteroatoms selected from nitrogen, oxygen, sulfur and mixtures thereof, and which rings contain no, one or more double bonds or may be non-aromatic, partly aromatic or wholly aromatic in character. The ring systems may be monocyclic, bicyclic or fused. Each “Het” group identified herein may be unsubstituted or substituted by one or more substituents selected from halo, cyano, nitro, oxo, lower alkyl (which alkyl group may itself be unsubstituted or substituted or terminated as defined herein) —OR¹⁹, —OC(O)R²⁰, —C(O)R²¹, —C(O)OR²², —N(R²³)R²⁴, —C(O)N(R²)R², —SR²⁹, —C(O)SR³⁰ or —C(S)N(R²⁷)R²⁶ wherein R¹⁹ to R³⁰ each independently represent hydrogen, unsubstituted or substituted aryl or lower alkyl (which alkyl group itself may be unsubstituted or substituted or terminated as defined herein). The term “Het” thus includes groups such as optionally substituted azetidinyl, pyrrolidinyl, imidazolyl, indolyl, furanyl, oxazolyl, isoxazolyl, oxadiazolyl, thiazolyl, thiadiazolyl, triazolyl, oxatriazolyl, thiatriazolyl, pyridazinyl, morpholinyl, pyrimidinyl, pyrazinyl, quinolinyl, isoquinolinyl, piperidinyl, pyrazolyl and piperazinyl. Substitution at Het may be at a carbon atom of the Het ring or, where appropriate, at one or more of the heteroatoms. “Het” groups may also be in the form of an N oxide.

The source of ruthenium may be present in any suitable amount. The phosphine compound may also be present in any suitable amount. Preferably, the molar ratio of ruthenium:phosphorous is from about 1:50 to about 2:1, preferably, from about 1:6 to about 1:1, most preferably about 1:2.

The molar ratio of ruthenium:phosphorous will be equal to the molar ratio of the ruthenium:phosphine compound for a monodentate phosphine; half the molar ratio of the ruthenium:phosphine compound for a bidentate phosphine; a third of the molar ratio of ruthenium:phosphine compound for a trivalent phosphine; and a quarter of the molar ratio of the ruthenium:phosphine compound for a tetravalent phosphine.

Any suitable reaction temperature may be used. However, it is preferable for the reaction of the present invention to be carried out at relatively low temperatures. A suitable range of temperatures in which the reaction may be carried out is from about 120° C. to about 250° C., preferably, between about 130° C. and about 200° C., more preferably between about 140° C. and about 180° C.

By the term “carboxylic acids and/or derivatives thereof” it is meant any compound containing a group of general formula II

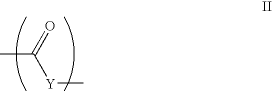

wherein Y may be a heteroatom such as O, N or S. Examples of compounds containing a group of general formula II include, but are not restricted to carboxylic acids, dicarboxylic acids, polycarboxylic acids, anhydrides, esters, amides and mixtures thereof. Preferably, the carboxylic acids and/or derivatives thereof of the present invention are selected from carboxylic acids, esters and/or amides, more preferably, amides are selected.

Suitable carboxylic acids are preferably any C₁-C₃₀ organic compound having at least one carboxylic acid group, more preferably any C₁ to C₁₆ organic compound having at least one carboxylic acid group. The organic compound may be optionally substituted as defined herein. The organic compound may be substituted with one or more of the following: hydroxy groups, C₁-C₄ alkoxy groups such as, for example, methoxy; amine or halide groups such as, for example Cl, I and Br. Examples of suitable carboxylic acids include, but are not restricted to, substituted and unsubstituted benzoic acids, acetic acids, propionic acids, valeric acids, butanoic acids, cyclohexylpropionic acids or nonanoic acids.

Suitable esters are preferably any C₁-C₃₀ organic compound having at least one ester group, more preferably any C₁ to C₁₆ organic compound having at least one ester group. The organic compound may be optionally substituted as defined herein. The organic compound may be substituted with one or more of the following: hydroxy groups, C₁-C₄ alkoxy groups such as, for example, methoxy; amine or halide groups such as, for example Cl, I and Br. Examples of suitable esters include, but are not restricted to, substituted and unsubstituted benzoates, methanoates, propanoates, pentanoates, butanoates, cyclohexylpropanoates or nonanoates.

Suitable amides are preferably any C₁-C₃₀ organic compound having at least one amide group, more preferably any C₁ to C₁₆ organic compound having at least one amide group. The organic compound may be optionally substituted as defined herein. The organic compound may be substituted with one or more of the following: hydroxy groups, C₁-C₄ alkoxy groups such as, for example, methoxy; amine or halide groups such as, for example Cl, I and Br. Examples of suitable amides include, but are not restricted to, substituted and unsubstituted benzamides, acetamides, propanamides, pentanamides, butanamides, cyclohexylpropanamides or nonamides. Preferred amides include butanamide and nonamides, for example N-phenylnonamide.

By organic compound, it is meant, unless otherwise specified, a compound which may, when there is a sufficient number of carbon atoms, be linear or branched, be saturated or unsaturated, be cyclic, polycyclic, acyclic or part cyclic/acyclic, be unsubstituted, substituted or terminated as defined herein and/or be interrupted by one or more (preferably less than 4) oxygen, sulphur, silicon atoms, or by silano or dialkylsilicon groups, or mixtures thereof.

It has advantageously been found that performing the hydrogenation reaction as described above in the presence of a source of ammonia leads to a highly selective reaction which favours the production of primary amines.

Thus, according to a third aspect of the present invention, there is provided a process for the hydrogenation of carboxylic acids and/or derivatives thereof comprising the steps of:
reacting said carboxylic acids and/or derivatives thereof with a source of hydrogen in the presence of a source of ammonia and a catalyst system, the said catalyst system obtainable by combining:
   a) a source of ruthenium; and
   b) a phosphine compound of general formula I:

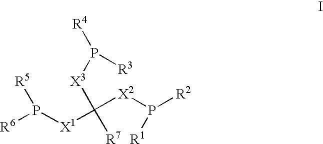

wherein X¹ to X³ and R¹ to R⁶ each independently represent lower alkyl or aryl, and R⁷ represents hydrogen, lower alkyl or aryl.

Preferably, the catalyst system is homogeneous.

According to a further aspect of the present invention, there is provided a use of ammonia in the production of primary amines by hydrogenation of carboxylic acids and/or derivatives thereof.

According to yet a further aspect of the present invention, there is provided a process for the production of primary amines comprising the steps of reacting a carboxylic acid and/or derivative thereof with a source of hydrogen and a source of ammonia in the presence of a catalyst system as described above.

It has been surprisingly found that the presence of the source of ammonia leads predominantly to the primary amine product. This is advantageous in the production of primary amine intermediates for further synthesis.

The ammonia used may be present in liquid, gaseous or aqueous form or any combination thereof. Preferably, the ammonia is present in either liquid or aqueous form.

When gaseous ammonia is used, it is preferably present in the gaseous phase of the reaction mixture at a partial pressure of between about 0.1 bar and about 25 bar, preferably between about 1 bar and about 15 bar, most preferably between about 2 bar and about 10 bar.

When liquid ammonia is added to the reaction mixture, it is preferably present is such an amount that the ratio of the volume of ammonia:volume of solvent is from about 1:100 to about 10:1, preferably from about 1:20 to about 5:1, most preferably from about 1:10 to about 2:1.

When aqueous ammonia is added to the reaction mixture, it is preferably added in an amount such that the ratio of the volume of ammonia:volume of solvent is as defined for liquid ammonia.

By “aqueous ammonia” is meant a solution of ammonia dissolved in water. The concentration of the ammonia in the aqueous solution may be in the range of 1% to 99% w/v, preferably, from about 10% to about 70% w/v, more preferably, from about 20% to about 50% w/v. A preferred aqueous ammonia solution may be obtained from Aldrich having a concentration of ammonia of about 34% w/v.

Preferably, when aqueous ammonia is used, it may be used in a suitable concentration and amount so that no further source of water need be added to the reaction mixture. However, the concentration of ammonia in the aqueous ammonia is also such that the desired concentration of ammonia is present in the reaction mixture and the resulting concentration of water is as required. The preferred concentration of ammonia in the total reaction mixture is between about 1% and about 30% w/v, preferably between about 2% and about 30% w/v, more preferably between about 5% and about 25% w/v.

For the avoidance of doubt w/v herein refers to grams per 100 ml.

Alternatively, the source of ammonia may be provided in solution with a different solvent. For example, ammonia may be provided in solution in alcohols such as methanol, ethanol and isopropanol; or in ethers such as dioxane.

The invention will now be described and illustrated by way of the following non-limiting examples and comparative examples.

### EXAMPLES

### Examples of 1-13 and Comparative Examples A-C

### Hydrogenation of N-Phenylnonamide

Examples 1-13 and comparative examples A-C show the hydrogenation of N-phenylnonamide in the presence of a ruthenium/phosphine catalyst.

### Example 1

1 g (4.28 mmoles) N-phenylnonamide was contacted with a catalyst system comprising a combination of Ru(acac)₃ (1 mole % relative to N-phenylnonamide) and 1,1,1-tris(diphenylphosphinomethyl)ethane, hereinafter referred to as “triphos” (2 mole % relative to N-phenylnonamide) and 10 ml of tetrahydrofuran solvent. Water was added to the reaction mixture so that the volume ratio of water:solvent therein was 1:10. The N-phenylnonamide was hydrogenated in the presence of the catalyst system under hydrogen gas at a pressure of 40 bar and at a temperature of 164° C. for a period of 14 hours. The reaction products were analysed by Gas Chromatography at the end of the reaction period. The results are summarised in Table 1.

The reaction resulted in full conversion of the amide, and a high selectivity (93%) to the amine product. The corresponding alcohol (7%) was obtained as a secondary product.

### Example 2

The method of example 1 was performed in the absence of additional water. The results are summarised in Table 1.

The results show that full conversion was obtained and only traces (1%) of alcohol were obtained. However, the catalyst was not stable under these conditions, so a minimum amount of water was included in subsequent reactions.

### Examples 3 to 6

The method of example 1 was carried out except that Ru(acac)₃ was replaced with various ruthenium catalyst precursors. The results are summarised in Table 1.

### Examples 7 to 9

The method of example 1 was carried out at various temperatures ranging from 100° C. to 140° C. The results are summarised in Table 1.

The results show that the hydrogenation of amides may be carried out at 140° C. without any apparent difference from actions at 164° C. but reducing the temperature to 120° C. resulted in a loss of selectivity, giving more alcohol from the amide, which is easier to reduce. Only alcohol (no amine) was produced at 100° C.

### Examples 10 to 13

The method of example 1 was carried out except that the tetrahydrofuran solvent was replaced with various alternative solvents. The results are summarised in Table 1.

The results show that toluene and ethereal solvents (diethyl ether and dioxane) yielded excellent conversion and selectivities similar to those obtained with tetrahydrofuran. The addition of aniline gave instability to the catalyst, resulting in a loss of both yield and selectivity.

### Comparative Example A

The method of example 1 was carried out except that the ruthenium triphos catalyst system was not used. The results are summarised in Table 1.

### Comparative Example B

The method of example 1 was carried out except that the ruthenium triphos catalyst system was replaced with Ru(acac)₃ alone. The results are summarised in Table 1.

### Comparative Example C

The method of example 1 was carried out except that the ruthenium triphos catalyst system was replaced with triphos alone. The results are summarised in Table 1.

The results of the comparative examples show that in the absence of ruthenium precatalyst, no conversion was obtained. The use of Ru(acac)₃ alone gives only a moderate yield of 61%.

TABLE 1

| | | Triphos | | T | Water:Solvent | Conversion | Secondary | Alcohol |
|---|---|---|---|---|---|---|---|---|
| Example | Ru compound (mole %) | (mole %) | Solvent | (° C.) | Ratio | (%) | amine (%) | (%) |
| 1 | Ru(acac)₃ (1%) | 2 | THF | 164 | 1:10 | 100 | 93 | 7 |
| 2 | Ru(acac)₃ (1%) | 2 | THF | 164 | 0 | 100 | 99 | 1 |
| 3 | RuCl₃ (1%) | 2 | THF | 164 | 1:10 | 100 | 64 | 36 |
| 4 | Ru(DMSO)₄ Cl₂ (1%) | 2 | THF | 164 | 1:10 | 100 | 90 | 10 |
| 5 | Ru(COD)Cl₂ (1%) | 2 | THF | 164 | 1:10 | 66 | 30 | 36 |
| 6 | [Ru₂(Triphos)₂Cl₃]Cl | — | THF | 164 | 1:10 | 100 | 95 | 5 |
| | (0.5%) | | | | | | | |
| 7 | Ru(acac)₃ (1%) | 2 | THF | 140 | 1:10 | 100 | 91 | 9 |
| 8 | Ru(acac)₃ (1%) | 2 | THF | 120 | 1:10 | 80 | 48 | 32 |
| 9 | Ru(acac)₃ (1%) | 2 | THF | 100 | 1:10 | 40 | 0 | 40 |
| 10 | Ru(acac)₃ (1%) | 2 | Et₂O | 164 | 1:10 | 100 | 92 | 8 |
| 11 | Ru(acac)₃ (1%) | 2 | Toluene | 164 | 1:10 | 100 | 93 | 9 |
| 12 | Ru(acac)₃ (1%) | 2 | Dioxane | 164 | 1:10 | 100 | 93 | 7 |
| 13 | Ru(acac)₃ (1%) | 2 | THF (+1 | 164 | 1:10 | 92 | 71 | 21 |
| | | | equivalent | | | | | |
| | | | aniline) | | | | | |
| A | — | — | THF | 164 | 1:10 | 0 | 0 | 0 |
| B | Ru(acac)₃ (1%) | — | THF | 164 | 1:10 | 61 | 57 | 4 |
| C | — | 2 | THF | 164 | 1:10 | 0 | 0 | 0 |

### Examples 14-21 and Comparative Examples D-E

### Hydrogenation of Butanamide to Produce a Primary Amine

Examples 14-21 show the hydrogenation of butanamide in the presence of ammonia to selectively produce the primary amine.

### Example 14

1 g butanamide was contacted with 10 ml of tetrahydrofuran solvent and a catalyst system comprising a combination of Ru(acac)₃ (1 mole % relative to butanamide) and triphos (2 mole % relative to butanamide). Water was added to the reaction mixture so that the volume ratio of water:solvent therein was 1:10. The butanamide was then hydrogenated under an atmosphere of hydrogen gas and gaseous ammonia. The ammonia was present at a partial pressure of 4 bar. The overall pressure of the hydrogen and ammonia gas was 40 bar. The reaction was carried out at a temperature of 164° C. for a period of 14 hours. The reaction products were analysed by Gas Chromatography at the end of the reaction period. The results are summarised in Table 2.

### Example 15

The method of example 14 was carried out except that the Ru(acac)₃ and triphos catalyst system was replaced by 91.5 mg (0.5 mole % relative to butanamide) [Ru₂(Triphos)₂Cl₃]Cl, and the atmosphere of gaseous ammonia was removed and replaced with liquid ammonia at a volume ratio of liquid ammonia:solvent of 1:2. The results are summarised in Table 2.

### Example 16

The method of example 15 was carried out, except that the volume ratio of liquid ammonia:solvent was increased to 1:1. The results are summarised in Table 2.

### Examples 17 to 20

The method of example 15 was carried out, except that the liquid ammonia was replaced with aqueous ammonia having a concentration of 34% w/v at various volume ratios to the solvent. The separate source of water was removed. The results are summarised in Table 2.

The results show that aqueous ammonia increased the selectivity of the reaction. However, a high excess of aqueous ammonia also increased the concentration of water, which increased the rate of hydrolysis of the amide resulting in a drop in selectivity.

### Example 21

The method of examples 17 to 20 was carried out with the aqueous ammonia present at a volume ratio of 1:1 with the solvent. The reaction was also carried out under an atmosphere of gaseous ammonia at a partial pressure of 4 bar. The results are summarised in Table 2.

### Comparative Example D

The method of example 14 was carried out in the absence of any source of ammonia. The results are summarised in Table 2.

### Comparative Example E

The method of comparative example D was carried out in the presence of a volume ratio of water:solvent of 1:100. The results are summarised in Table 2.

Comparative examples D and E show that hydrogenation in the absence of ammonia gave a low selectivity. However, no primary amine was obtained.

TABLE 2

| | | | Aqueous | Liquid | | Primary | | Tertiary | | |
|---|---|---|---|---|---|---|---|---|---|---|
| | Water:Solvent | P(NH₃) | Ammonia:THF | NH₃:THF | Conversion | amine | Secondary | amine | Secondary | Alcohol |
| Example | Ratio | bar | ratio | ratio | (%) | (%) | amine (%) | (%) | amide (%) | (%) |
| 14 | 1:10 | 4 | — | — | 100 | 32 | 20 | 15 | 2 | 31 |
| 15 | 1:10 | — | — | 1:2 | 100 | 44 | 38 | 0 | 10 | 8 |
| 16 | 1:10 | — | — | 1:1 | 59 | 36 | 6 | 0 | 14 | 3 |
| 17 | — | — | 3:10 | — | 100 | 78 | 0 | 0 | 10 | 12 |
| 18 | — | — | 1:2 | — | 100 | 85 | 0 | 0 | 0 | 15 |
| 19 | — | — | 7:10 | — | 100 | 85 | 0 | 0 | 0 | 15 |
| 20 | — | — | 1:1 | — | 100 | 73 | 0 | 0 | 2 | 25 |
| 21 | — | 4 | 1:1 | — | 100 | 75 | 0 | 0 | 0 | 25 |
| D | 1:10 | — | — | — | 100 | 0 | 46 | 53 | Traces | Traces |
| E | 1:100 | — | — | — | 100 | 0 | 48 | 51 | Traces | Traces |

### Examples 22-25

### Hydrogenation of Nonanoic Acid

Examples 22-25 show a direct synthesis route from nonanoic acid to the desired primary amine. The synthesis involves generation of the primary amide in situ from the acid and ammonia, followed by the subsequent hydrogenation of the primary amide to the primary amine.

### Example 22

1 ml nonanoic acid was contacted with liquid ammonia in the presence of 10 ml tetrahydrofuran solvent and 0.5 mole % [Ru₂(Triphos)₂Cl₃]Cl relative to nonanoic acid. The liquid ammonia was present at a volume ratio of 1:2 with the solvent. Water was added to the reaction mixture at a volume ratio of 1:10 with the solvent. The acid was hydrogenated under an atmosphere of hydrogen gas at a pressure of 40 bar and at a temperature of 164° C. for a period of 14 hours. The reaction products were analysed by Gas Chromatography at the end of the reaction period. The results are summarised in Table 3.

### Example 23

The method of example 22 was carried out except the volume ratio of liquid ammonia:solvent was increased to 1:1.

### Example 24

The method of example 22 was carried out except that the source of water was removed and the liquid ammonia was replaced with aqueous ammonia having a concentration of 34% w/v.

### Example 25

The method of example 24 was carried out except the volume ratio of aqueous ammonia:solvent was increased to 1:1.

TABLE 3

| | | Aqueous | Liquid | | Primary | | Tertiary | | |
|---|---|---|---|---|---|---|---|---|---|
| | Water:Solvent | Ammonia:THF | NH₃:THF | Conversion | amine | Secondary | amine | Secondary | Alcohol |
| Example | ratio | ratio | ratio | (%) | (%) | amine (%) | (%) | amide (%) | (%) |
| 22 | 1:10 | — | 1:2 | 100 | 15 | 47 | 0 | 35 | 3 |
| 23 | 1:10 | — | 1:1 | 100 | 23 | 22 | 0 | 55 | 0 |
| 24 | — | 1:2 | — | 100 | 49 | 37 | 0 | 9 | 5 |
| 25 | — | 1:1 | — | 100 | 41 | 31 | 0 | Traces | 28 |

Using the process of the present invention, it has surprisingly been found that hydrogenation reactions using a specific, homogeneous catalyst system lead to a highly selective conversion of an amide to the desired amine product.

Furthermore, it has also been found that a primary amine may be selectively produced in high yield from the hydrogenation of an amide in the presence of the homogeneous catalyst system and ammonia.

The conversion and selectivity of the hydrogenation of amides may be further increased by the use of low levels of water and/or by performing the reaction under low pressures.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A process for the hydrogenation of amides comprising the steps of:—
reacting said amide with a source of hydrogen in the presence of a catalyst system, the said catalyst system obtainable by combining:
(a) a source of ruthenium, and
(b) a phosphine compound of general formula I: 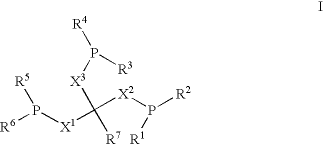
wherein X¹ to X³ and R¹ to R⁶ each independently represent lower alkyl or aryl, and R⁷ represents hydrogen, lower alkyl or aryl,
wherein the hydrogenation reaction is carried out in the presence of a low concentration of water wherein the ratio of moles of water:moles of ruthenium present at the start of a batch reaction or during a continuous reaction is up to 2000:1 or the hydrogenation reaction is carried out in the absence of water.

2. A process for the hydrogenation of carboxylic acids and/or derivatives thereof comprising the steps of:—
reacting said carboxylic acids and/or derivatives thereof with a source of hydrogen in the presence of a catalyst system, the said catalyst system obtainable by combining:
(a) a source of ruthenium, and
(b) a phosphine compound of general formula I: 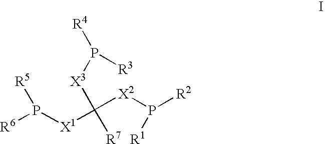
wherein X¹ to X³ and R¹ to R⁶ each independently represent lower alkyl or aryl, and
R⁷ represents hydrogen, lower alkyl or aryl, wherein the reaction is carried out at a low pressure.

3. A process for the hydrogenation of carboxylic acids and/or derivatives thereof comprising the steps of:
reacting said carboxylic acids and/or derivatives thereof with a source of hydrogen in the presence of a source of ammonia and a catalyst system, the said catalyst system obtainable by combining:
a) a source of ruthenium; and
b) a phosphine compound of general formula I: 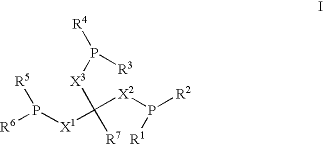
wherein X¹ to X³ and R¹ to R⁶ each independently represent lower alkyl or aryl, and R⁷ represents hydrogen, lower alkyl or aryl.

4. The process according to claim 1, wherein the catalyst system is homogeneous.

5. The process according to claim 1, wherein the step of reacting said amides or carboxylic acids and/or derivatives thereof with a source of hydrogen in the presence of a homogenous catalyst system is carried out in the presence of at least one solvent.

6. The process according to claim 2, wherein the ratio of moles of water:moles of ruthenium present at the start of a batch reaction or during a continuous reaction is up to about 2500:1.

7. The process according to claim 1 wherein, the ratio of moles of water:moles of ruthenium present at the start of a batch reaction or during a continuous reaction is at least about 50:1.

8. The process according to claim 1, wherein the reaction is carried out under a pressure of up to about 6.5×10⁶ Pa.

9. The process according to claim 1, wherein X¹ to X³ in formula I each independently represent a divalent bridging group.

10. The process according to claim 1, wherein specific examples of phosphine compounds of general formula I include, but are not limited to, tris-1,1,1-(diphenylphosphinomethyl)methane, tris-1,1,1-(diphenylphosphinomethyl)-ethane, tris-1,1,1-(diphenylphosphinomethyl)propane, tris-1,1,1-(diphenylphosphinomethyl)butane, tris-1,1,1-(diphenylphosphinomethyl)2-ethane-butane, tris-1,1,1-(diphenylphosphinomethyl)2,2-dimethylpropane, tris-1,1,1-(dicyclohexylphosphinomethyl)ethane, tris-1,1,1-(dimethylphosphinomethyl)ethane and tris-1,1,1-(diethylphosphinomethyl)ethane.

11. The process according to claim 1, wherein the molar ratio of ruthenium:phosphorous is from about 1:50 to about 2:1.

12. The process according to claim 3, wherein the ammonia used is present in liquid, gaseous or aqueous form or any combination thereof.

13. The process according to claim 3, wherein when gaseous ammonia is used, it is present in the gaseous phase of the reaction mixture at a partial pressure of between about 0.1 bar and about 25 bar.

14. The process according to claim 3, wherein when liquid ammonia is added to the reaction mixture, it is present in such an amount that the ratio of the volume of ammonia:volume of solvent is from about 1:100 to about 10:1.

15. The process according to claim 3, wherein when aqueous ammonia is added to the reaction mixture, it is added in an amount such that the ratio of the volume of ammonia:volume of solvent is as defined for liquid ammonia.

16. The use of ammonia in the production of primary amines by hydrogenation of carboxylic acids and/or derivatives thereof.

17. A process for the production of primary amines comprising the steps of reacting a carboxylic acid and/or derivative thereof with a source of hydrogen and a source of ammonia in the presence of a catalyst system as claimed in claim 1.

18. (canceled)

19. (canceled)

20. (canceled)
